(19) United States
(12) Patent Application Publication
(10) Pub. No.: US 2007/0093815 A1
(43) Pub. Date: Apr. 26, 2007

(54) Dynamic spinal stabilizer

(76) Inventors: Ronald Callahan II, Naugatuck, CT (US); Ernest Corrao, Bethel, CT (US); Stephen Maguire, Shelton, CT (US); Stephen Santangelo, New Hartford, CT (US)

Correspondence Address: McCarter & English, LLP;Financial Centre, Suite 304A, 695 East Main Street, Stamford, CT, 06901-2138, US

(21) Appl. No.: 11/247,860

(22) Filed: Oct. 11, 2005

(51) Int. Cl. A61F 2/30

(52) U.S. Cl. 606/61

(57) ABSTRACT

An elongated member forming a spinal support rod is implantable adjacent the spine of a patient and includes an axial span or spans for spanning respective spinal levels to promote efficacious spinal support/stabilization. The axial span manifests a double helical geometry. The axial span has a rod-like profile of a diameter similar to conventional spinal support rods used for lumbar spinal fusion, and provides for use across multiple spinal levels and with multiple adjustable attachment points for associated spine attachment devices, such as pedicle screws, to accommodate different patient anatomies.

### BACKGROUND

1. Technical Field

The present disclosure relates to devices, systems and methods for spinal stabilization. More particularly, the present disclosure relates to devices, systems and methods for providing dynamic stabilization to the spine via the use of elongated members spanning one or more spinal levels.

2. Background Art

Each year, over 200,000 patients undergo lumbar fusion surgery in the United States. While fusion is a well-established procedure that is effective about seventy percent of the time, there are consequences even to successful fusion procedures, including a reduced range of motion and an increased load transfer to adjacent levels of the spine, which may accelerate degeneration at those levels. Further, a significant number of back-pain patients, estimated to exceed seven million in the U.S., simply endure chronic low-back pain, rather than risk procedures that may not be appropriate or effective in alleviating their symptoms.

New treatment modalities, collectively called motion preservation devices, are currently being developed to address these limitations. Some promising therapies are in the form of nucleus, disc or facet replacements. Other motion preservation devices provide dynamic internal stabilization of the injured and/or degenerated spine, e.g., the Dynesis stabilization system (Zimmer, Inc.; Warsaw, Ind.) and the Graf Ligament. A major goal of this concept is the stabilization of the spine to prevent pain while preserving near normal spinal function.

In general, while great strides are currently being made in the development of motion preservation devices, the use of such devices is not yet widespread. One reason that this is so is the experimental nature of most such devices. For example, to the extent that a given motion device diverges, whether structurally or in its method of use or implementation, from well-established existing procedures such as lumbar fusion surgery, considerable experimentation and/or testing is often necessary before such a device is given official approval by governmental regulators, and/or is accepted by the medical community as a safe and efficacious surgical option.

With the foregoing in mind, those skilled in the art will understand that a need exists for spinal stabilization devices, systems and methods that preserve spinal motion while at the same time exhibiting sufficient similarity to well-established existing spinal stabilization devices, systems and methods so as encourage quick adoption/approval of the new technology. These and other needs are satisfied by the disclosed devices, systems and methods that include elongated members for implantation across one or more levels of the spine.

### SUMMARY OF THE PRESENT DISCLOSURE

According to the present disclosure, advantageous devices, systems, kits for assembly, and/or methods for dynamic stabilization are provided. According to exemplary embodiments of the present disclosure, the disclosed devices, systems, kits and methods include an elongated member, e.g., a spinal support rod, that is configured and dimensioned for implantation adjacent the spine of a patient so as to promote efficacious spinal stabilization. The disclosed elongate member further manifests (at least in part) a double helical geometry.

According to exemplary embodiments of the present disclosure, the elongated member includes an axial span that extends in an axial direction across at least one spinal level to promote efficacious spinal stabilization thereacross, and that manifests a double helical geometry. In some such embodiments, the elongated member is configured and dimensioned for implantation adjacent the spine such that at least two axial spans of the elongated member extend across respective spinal levels of the spine to promote efficacious spinal stabilization thereacross. Both such axial spans manifest (at least in part) a double helical geometry. In some such embodiments, the axial span has a rod-like profile and is adapted to be coupled to the spine of the patient via attachment to conventional spine attachment devices configured for coupling conventional support rods, such as solid, relatively inflexible spinal support rods used in conjunction with spinal fusion assemblies, to the spine. Such rod-like profile can include a diameter in a range of from about 5.5 mm to 6.35 mm, although alternative dimensions and/or dimensional ranges may be employed, and the axial span can be adapted to permit pedicle screws or other mounting structures (e.g., hooks, plates, cemented stems and the like) to be attached to the elongated member at multiple points along the length of the axial span so as to accommodate a range of different patient anatomies and spinal height levels. Further with respect to some exemplary embodiments, the axial span is axially rigid as against axial forces arrayed in compression and/or tension. Still further with respect to some exemplary embodiments, the double helical geometry manifested by the axial span includes two peripheral surfaces disposed substantially diametrically opposite each other, and two inclined surfaces extending transversely between the two peripheral surfaces and oriented substantially parallel to each other.

Further, in some such embodiments, the double helical geometry manifested by the axial span permits the axial span to bend, flex or deflect along any and substantially all transverse directions while providing efficacious spinal stabilization across the at least one spinal level during at least one of spinal flexion, spinal extension, spinal lateral bending, and spinal axial rotation. The axial span provides efficacious spinal stabilization across the at least one spinal level during: a) spinal flexion in which the spinal level defines an anterior bend of at least approximately seven degrees; b) spinal extension in which the spinal level defines a posterior bend of at least approximately seven degrees; and c) spinal bending in which said spinal level defines a lateral bend of at least approximately seven degrees.

According to further embodiments of the present disclosure, a surgically implantable spinal support rod is provided that includes an axial span that extends in an axial direction so as to span at least one spinal level, wherein the axial span manifests (at least in part) a double helical geometry. In some such embodiments, the double helical geometry manifested by the axial span includes two peripheral surfaces disposed substantially diametrically opposite each other, and two helically inclined surfaces extending transversely between the two peripheral surfaces and oriented substantially parallel to each other. In some other such embodiments, the axial span has a rod-like profile, and is adapted to be coupled to the spine of the patient via attachment to conventional spine attachment devices (e.g., pedicle screws, hooks, plates, stems and the like) configured for coupling conventional support rods to the spine for purposes of spinal fusion or other spinal procedures. The rod-like profile of the axial span can include a diameter in a range of from about 5.5 mm to about 6.35 mm, although alternative dimensions and/or dimensional ranges may be employed.

In accordance with still further embodiments of the present disclosure, a kit for assembling a dynamic spinal support system is provided. Such kit includes a spinal support rod having an axial span extending in an axial direction so as to span at least one spinal level, and manifesting a double helical geometry. Such kit also includes a plurality of spine attachment devices (e.g., pedicle screws, hooks, plates, stems, or combinations thereof) respectively attachable to the axial span so as to couple the spinal support rod to the spine of a patient across the spinal level.

The elongated elements/spinal support rods of the present disclosure, and/or the spinal stabilization devices/systems of the present disclosure incorporating such elongated elements/spinal support rods, advantageously include one or more of the following structural and/or functional attributes:

Spine surgery patients whose conditions indicate that they would benefit from retaining at least some spinal motion in flexion, extension, and/or axial rotation may be fitted with a dynamic spinal stabilization device/system as disclosed herein rather than undergo procedures involving substantial immobilization as between adjacent vertebrae;

The elongated members/spinal support rods in accordance with the present disclosure are compatible (e.g., by virtue of standard diameter sizing, substantial dimensional/diametrical stability, and/or rigidity in axial tension and axial compression, etc.) with most rod attachment hardware presently being implanted in conjunction with lumbar fusion surgery or other spinal procedures, enhancing the likelihood of quick adoption by the medical community and/or governmental regulatory approval;

The elongated members/spinal support rods disclosed herein are adaptable to pedicle screw attachment or other conventional mounting apparatus (e.g., mounting hooks, plates, stems and the like), can be used across one or more spinal levels, permit at least approximately seven degrees of spinal extension, and spinal flexion, and/or spinal lateral bending as between adjacent spinal vertebrae, and allow for adjustable attachment points along their axial lengths to accommodate differing patient anatomies.

Advantageous spine stabilization devices, systems, kits for assembling such devices or systems, and methods may incorporate one or more of the foregoing structural or functional attributes. Thus, it is contemplated that a system, device, kit and/or method may utilize only one of the advantageous structures/functions set forth above, or all of the foregoing structures/functions, without departing from the spirit or scope of the present disclosure. Stated differently, each of the structures and functions described herein is believed to offer benefits, e.g., clinical advantages to clinicians or patients, whether used alone or in combination with others of the disclosed structures/functions.

Additional advantageous features and functions associated with the devices, systems, kits and methods of the present disclosure will be apparent to persons skilled in the art from the detailed description which follows, particularly when read in conjunction with the figures appended hereto. Such additional features and functions, including the structural and mechanistic characteristics associated therewith, are expressly encompassed within the scope of the present invention.

### BRIEF DESCRIPTION OF THE DRAWINGS

To assist those of ordinary skill in the art in making and using the disclosed devices, systems and methods, reference is made to the appended figures, in which:

FIGS. 1, 2 and 3 are respective side, top, and end views of a dynamic spinal stabilization device/system implanted into the spine of a patient, in accordance with a first embodiment of the present disclosure;

FIG. 4 is a downward perspective view of a flexible elongated member of the spinal stabilization device/system of FIGS. 1-3;

FIG. 5 is a side illustration of the flexible elongated member of FIG. 4;

FIG. 6 is a cross-sectional view of the flexible elongated member of FIGS. 4 and 5, taken along section line 6-6 in FIG. 5;

FIG. 7 is a side illustration of the spinal stabilization device/system of FIGS. 1-3, wherein the patient is in spinal flexion;

FIG. 8 is a side illustration of the spinal stabilization device/system of FIGS. 1-3, wherein the patient is in spinal extension;

FIGS. 9 and 10 are top views of the spinal stabilization device/system of FIGS. 1-3, wherein the spine of the patient is bending to the left, and to the right, respectively; and

FIGS. 11 and 12 are end views of the spinal stabilization device/system of FIGS. 1-3, wherein the spine of the patient is twisting to the right, and to the left, respectively.

### DESCRIPTION OF EXEMPLARY EMBODIMENTS

The present disclosure provides advantageous devices, systems and methods for providing dynamic spinal stabilization. More particularly, the present disclosure provide elongated members in the form of rods that are suitable for surgical implantation across multiple spinal levels for purposes of support and stabilization in flexion, extension, and/or axial rotation, and that are also laterally flexible so as to provide a range of motion in spinal flexion, extension, and/or axial rotation.

The exemplary embodiments disclosed herein are illustrative of the advantageous spinal stabilization devices/systems and surgical implants of the present disclosure, and of methods/techniques for implementation thereof. It should be understood, however, that the disclosed embodiments are merely exemplary of the present invention, which may be embodied in various forms. Therefore, the details disclosed herein with reference to exemplary dynamic stabilization systems and associated methods/techniques of assembly and use are not to be interpreted as limiting, but merely as the basis for teaching one skilled in the art how to make and use the advantageous spinal stabilization systems and alternative surgical implants of the present disclosure.

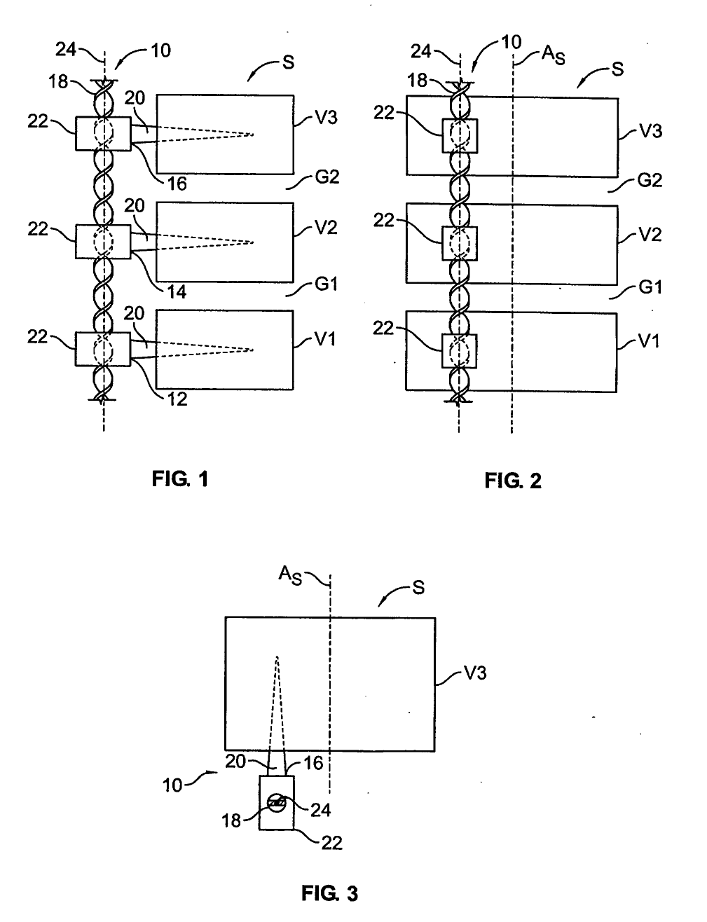

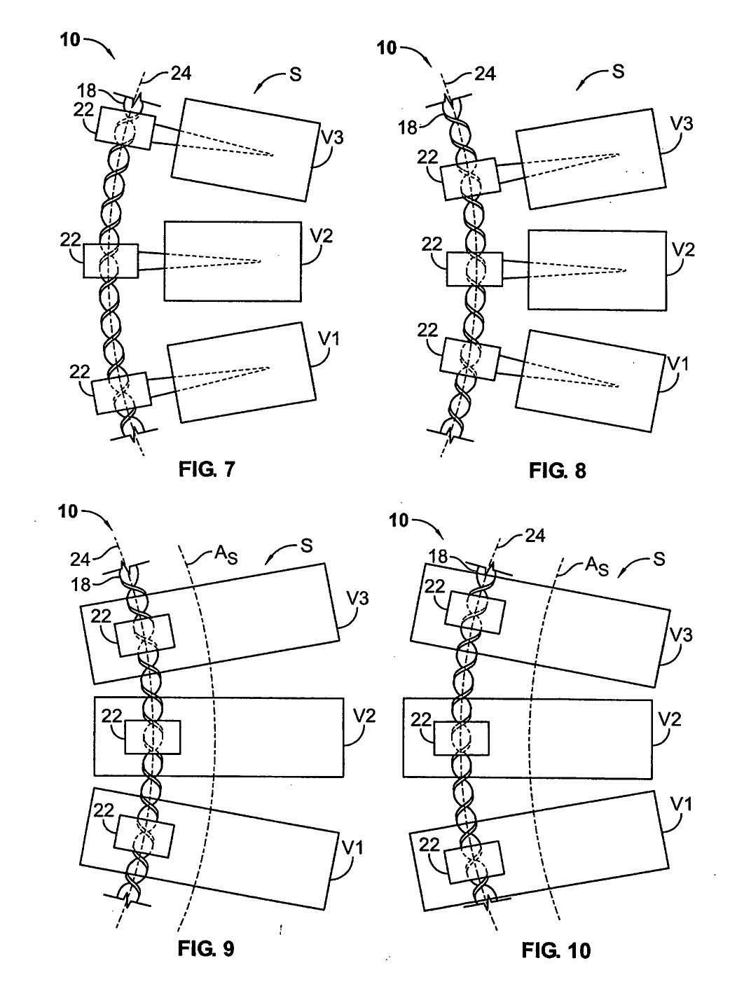

With reference to FIGS. 1-3, a dynamic spinal stabilization system 10 is shown implanted into and/or relative to the spine S of a patient, such spine S being rendered schematically in FIGS. 1-3 (as well as in FIGS. 7-12, the details of which are described more fully hereinbelow) in the form of three adjacent sequential vertebrae V1, V2 and V3 separated by corresponding intervertebral gaps G1 and G2. The dynamic stabilization system 10 is attached to the spine S along one lateral side thereof as defined by a bilateral axis of symmetry Aₛ thereof (another dynamic spine stabilization system 10 (not shown) can be attached to the spine S along the other lateral side thereof as desired and/or as necessary). The spinal stabilization system 10 includes three spine attachment elements 12, 14, 16, and an elongated member 18 spanning all of the vertebrae V1, V2, V3 (e.g., at least insofar as the gaps G1, G2 therebetween).

Each of the spine attachment elements 12, 14, 16 of the spinal stabilization system 10 includes an attachment extension 20 (depicted at least partially schematically) and an attachment member 22 (also depicted at least partially schematically). The spine attachment elements 12, 14, 16 are securely affixed to the respective vertebrae V1, V2, V3 via respective ends of the attachment extensions 20 being embedded within corresponding voids in the tissue of the respective vertebrae V1, V2, V2, and being securely retained therein (i.e., so as to prevent the attachment extensions 20 from being pulled out of their respective voids, or rotated with respect thereto, whether axially or otherwise). The attachment extensions 20 are embedded into and/or retained within their respective vertebral voids via suitable conventional means, such as a helical thread and/or a helically-shaped inclined plane formed on the respective attachment extension 20, a biocompatible adhesive, or other means of embedding and/or retention. The attachment extensions 20 form respective parts of and/or are mounted with respect to respective pedicle screws of conventional structure and function in accordance with at least some embodiments of the present disclosure. The attachment extensions 20 form parts of other types of structures than that of conventional pedicle screws in accordance with some other embodiments of the present disclosure, e.g., mounting hooks, plates, stems and the like.

The attachment extensions 20 and attachment members 22 of the spine attachment elements 12, 14, 16 are attached or coupled with respect to each other at respective ends of the attachment extensions 20 opposite the ends thereof that are embedded within the tissue of the respective vertebrae V1, V2, V3. Movable joints are advantageously formed at the points where the attachment extensions 20 and the attachment members 22 are attached/coupled. In at least some embodiments of the present disclosure, the ends of the attachment extensions 20 that are attached/coupled with respect to the respective attachment members 22 include respective pedicle screw heads of conventional structure and function. In some other embodiments of the present disclosure, such ends include types of structure other than that of conventional pedicle screw heads (e.g., hooks, plates, stems and the like). The movable joints formed between the attachment extensions 20 and the attachment members 22 may advantageously permit relatively unconstrained relative rotation (e.g., global rotation) therebetween, as well as at least some rotation of each attachment member 22 about an axis defined by the corresponding attachment extension 20. The structure and function of the movable joints between the attachment extensions 20 and the attachment members 22 of the respective spine attachment elements 12, 14, 16 will be described in greater detail hereinafter.

The attachment members 22 of the spine attachment elements 12, 14, 16 are generally configured and dimensioned so as to be operatively coupled to known spinal support rods (not shown) such as spinal support rods of conventional structure and having a standard diameter (e.g., from about 5.5 mm to about 6.35 mm) and that are commonly used in connection with lumbar fusion surgery and/or other spinal stabilization procedures. For example, in accordance with some embodiments of the present disclosure, each of the attachment members 22 is configured to couple to a conventional spinal support rod (not shown) so as to prevent relative movement between the attachment members 22 and the rod in a direction transverse (e.g., perpendicular) to the rod's axial direction of extension, and at least one of the attachment members 22 is further adapted to prevent relative movement between such attachment member 22 and the rod along the rod's axial direction of extension. The particular structures and characteristic functions of the attachment members 22 of the spine attachment elements 12, 14, 16 are discussed in greater detail hereinafter.

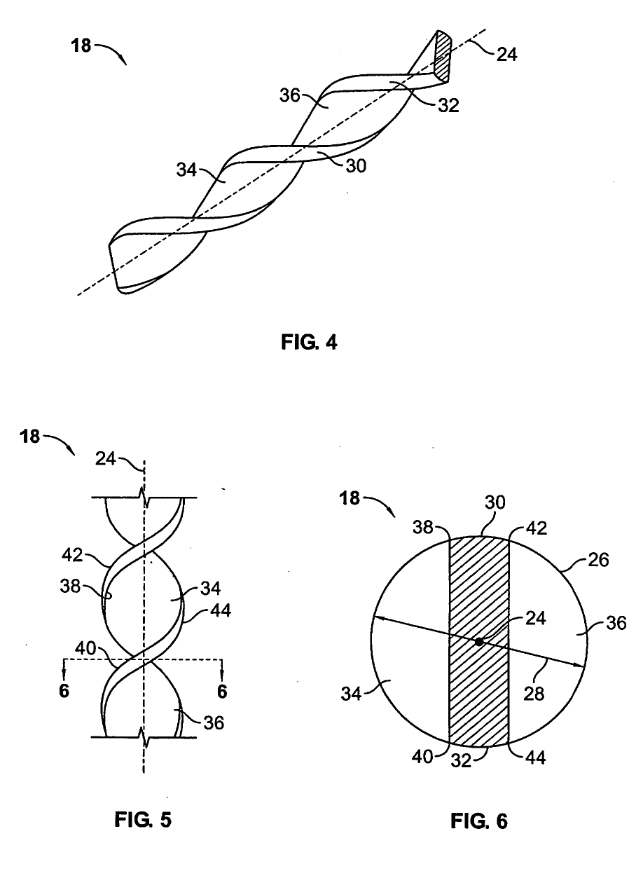

Referring now to FIGS. 4-6, the exemplary elongated member 18 of the spinal stabilization system 10 (FIG. 1) includes an axis 24 defined by an axial/longitudinal direction along which the elongated member 18 characteristically extends. As shown in FIG. 6, the elongated member 18 has an outer perimeter 26 in end view that has a substantially circular shape. The circular outer perimeter 26 defines a basic diameter 28 of the elongated member of an extent consistent with that of conventional spinal stabilization rods (e.g., an extent in a diameter range of from about 5.5 mm to about 6.35 mm, although other dimensions/dimensional ranges may be employed) such that the elongated member 18 is compatible with hardware designed to couple to conventional spinal stabilization rods and associated anatomical features and criteria. Accordingly, and referring again to FIGS. 1-3, the elongated member 18 is compatible with the spine attachment elements 12, 14, 16. More particularly, the elongated member is coupled to the attachment members 22 of the spine attachment elements 12, 14, 16 such that transverse movement of the elongated member 18 relative to the respective attachment members 22 is substantially limited and/or prevented. This is consistent with the support and stabilization function (described in greater detail hereinafter) of the elongated member 18 with respect to the spine S.

With respect to at least one of the attachment members 22, the elongated member 18 is coupled thereto such that motion/translation of the elongated member 18 in the axial direction (i.e., in the direction of the axis 24) relative to such attachment member(s) is substantially limited and/or prevented. This ensures that the elongated member 18 is prevented from freely and/or uncontrollably moving/translating in the axial direction with respect to the spine attachment elements 12, 14, 16 in the context of the overall spinal stabilization system 10. Moreover, in accordance with the embodiment of the present disclosure illustrated in FIGS. 1-6, the global joints formed between the attachment members 22 and the attachment extensions 20 of the respective spine attachment elements 12, 14, 16 allow the attachment members 22 to rotate to some degree along with the elongated member 18 relative to the spine S. The significance of such aspects of the connection between the elongated member 18 and the spine attachment elements 12, 14, 16 is described more fully hereinafter.

The elongated member 18 is also similar to conventional spinal stabilization rods in that it is substantially dimensionally stable in the radial direction (e.g., transversely/perpendicularly relative to the axial direction of extension of the elongated member 18 as represented by the axis 24). Accordingly, the elongated member 18 is capable of withstanding radially-directed compression forces imposed by any and/or all of the attachment members 22 either during the process of implanting the elongated member 18 along the spine S (e.g., in response to clamping forces imposed by any attachment member 22 on the elongated member 18) or during in situ use of the spinal stabilization system 10 (the details of such use being described more fully hereinafter). In accordance with at least some embodiments of the present disclosure, the elongated member 18 is of a continuous, unitary structure made from a biocompatible metallic structural material, such as a titanium or stainless steel alloy. Further with respect to such embodiments, the material and structural aspects of the elongated member 18 described herein render the elongated member 18 substantially rigid in axial tension, as well as substantially incompressible when subjected to axially-directed compression forces.

Still referring to FIGS. 4-6, the elongated element 18 includes four axially-extending external surfaces, to wit: 1) a first peripheral edge surface 30; 2) a second peripheral edge surface 32 disposed diametrically opposite the axis 24 from the first peripheral edge surface 30; 3) a first inclined surface 34 extending between the first and second peripheral edge surfaces 30, 32; and 4) a second inclined surface 36 extending between the first and second peripheral edge surfaces 30, 32 and disposed diametrically opposite the axis 24 from the first inclined surface 34. According to exemplary embodiments of the present disclosure, all points on the first and second peripheral edge surfaces 30, 32 are equidistant from the axis 24, and each of the first and second peripheral edge surfaces 30, 32 extends radially around the axis 24, such that the first and second peripheral edge surfaces 30, 32 collectively define the circular outer perimeter 26 of the elongated member 18, as well as the basic diameter 28 thereof Further, the first and second peripheral edge surfaces 30, 32 also extend axially/longitudinally, such that the first and second peripheral edge surfaces 30, 32 collectively define the axis 24, and so as to form a double helix. The double helix is generally characterized in that the rate of rotation of the first and second peripheral edge surfaces 30, 32 per unit length of the elongated element 18 is substantially consistent along the axial length of the elongated element 18 at approximately 6-12 mm per complete radial turn.

The first inclined surface 34 extends in a transversely straight linear direction between an edge line 38 associated with the first peripheral edge surface 30 and an edge line 40 associated with the second peripheral edge surface 32. The second inclined surface 36 extends in a transversely straight linear direction between an edge line 42 associated with the first peripheral edge surface 30 and parallel to the edge line 38 thereof, and an edge line 44 of the second peripheral edge surface 32 and parallel to the edge line 40 thereof The aforementioned straight/linear transverse directions of extension of the first and second inclined surfaces 34, 36 exist along substantially the entire length of the elongated element 18, and also remain substantially parallel to each other (see, e.g., FIG. 6) at all points along such length. Accordingly, the first and second inclined surfaces 34, 36 substantially completely track the regular helical path defined by the first and second peripheral edge surfaces 30, 32, are substantially parallel to each other.

In operation, e.g., when incorporated in the spinal stabilization system 10 adjacent the spine S of a patient as described hereinabove, the elongated member 18 is capable of providing support to the spine S in any one or more, or all, of spinal flexion, spinal extension, and spinal axial rotation. As may be seen by comparing FIGS. 1 and 7, the elongated member 18 of the spinal stabilization system 10 is sufficiently flexible to bend, flex or deflect from a substantially linear configuration (FIG. 1) to a configuration in which the elongated member 18 includes an anterior bend (FIG. 7), while being also sufficiently stiff to provide ample support to the vertebrae V1, V2, V3 of the spine S against undue spinal flexion, as determined by the anatomy and/or particular medical condition of the patient. In accordance with some embodiments of the present disclosure, the elongated member 18 is dimensioned and configured so as to permit such spinal flexion between adjacent vertebrae (e.g., between vertebrae V1 and V2, or between vertebrae V2 and V3) to an extent of at least approximately seven degrees.

As may be seen by comparing FIGS. 1 and 7, the elongated member 18 of the spinal stabilization system 10 is sufficiently flexible to bend, flex or deflect from a substantially linear configuration (FIG. 1) to a configuration in which the elongated member 18 includes an anterior bend (FIG. 7), while being also sufficiently stiff to provide clinically efficacious support to the vertebrae V1, V2, V3 of the spine S against undue spinal flexion, as determined by the anatomy and/or particular medical condition of the patient. In accordance with some embodiments of the present disclosure, the elongated member 18 is dimensioned and configured so as to permit such spinal flexion between adjacent vertebrae (e.g., between vertebrae V1 and V2, or between vertebrae V2 and V3) to an extent of at least approximately seven degrees.

As may be seen by comparing FIGS. 1 and 8, the elongated member 18 of the spinal stabilization system 10 is sufficiently flexible to bend, flex or deflect from a substantially linear configuration (FIG. 1) to a configuration in which the elongated member 18 includes a posterior bend (FIG. 8), while being also sufficiently stiff to provide clinically efficacious support to the vertebrae V1, V2, V3 of the spine S against undue spinal extension, as determined by the anatomy and/or particular medical condition of the patient. In accordance with some embodiments of the present disclosure, the elongated member 18 is dimensioned and configured so as to permit such spinal extension between adjacent vertebrae (e.g., between vertebrae V1 and V2, or between vertebrae V2 and V3) to an extent of at least approximately seven degrees.

As may be seen by comparing FIG. 2 to FIGS. 9 and 10, respectively, the elongated member 18 of the spinal stabilization system 10 is sufficiently flexible to bend, flex or deflect from a substantially linear configuration (FIG. 2) to a configuration in which the elongated member 18 includes a leftward bend (FIG. 9) or a rightward bend (FIG. 10) as reflected in the respective curves in the axis of symmetry Aₛ of the spine S, while being also sufficiently stiff to provide support to the vertebrae V1, V2, V3 of the spine S against undue spinal lateral bending, as determined by the anatomy and/or particular medical condition of the patient. In accordance with some embodiments of the present disclosure, the elongated member 18 is dimensioned and configured so as to permit such spinal lateral bending between adjacent vertebrae (e.g., between vertebrae V1 and V2, or between vertebrae V2 and V3) to an extent of at least approximately seven degrees.

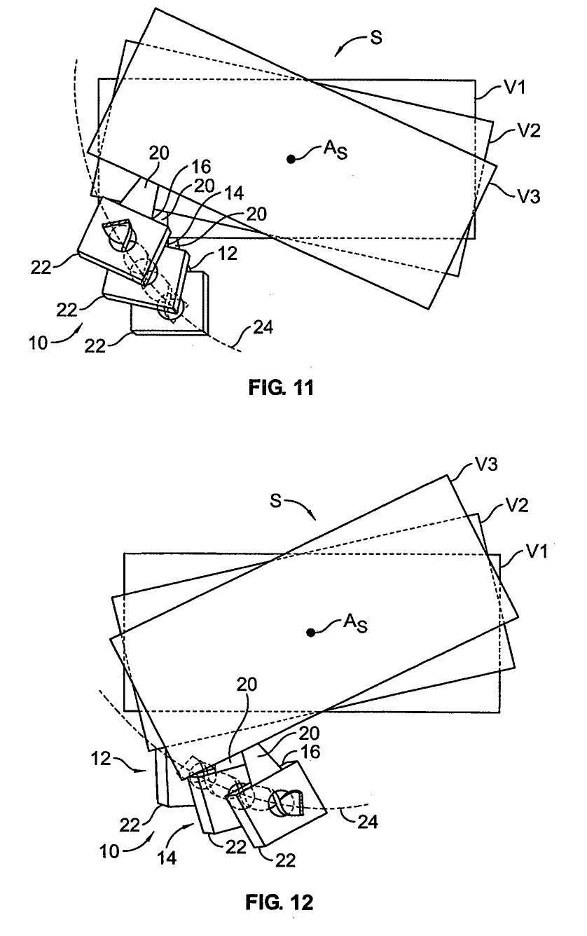

As may be seen by comparing FIG. 3 to FIGS. 11 and 12, respectively, the elongated member 18 of the spinal stabilization system 10 is sufficiently flexible to bend, flex or deflect from a substantially linear configuration (FIG. 3) to a configuration in which the elongated member 18 includes a leftward helical bend (FIG. 11) or a rightward helical bend (FIG. 12) about the axis of symmetry Aₛ of the spine S, while being also sufficiently stiff to provide ample support to the vertebrae V1, V2, V3 of the spine S against undue axial rotation, as determined by the anatomy and/or particular medical condition of the patient. In accordance with some embodiments of the present disclosure, the elongated member 18 is dimensioned and configured so as to permit such axial rotation between adjacent vertebrae (e.g., between vertebrae V1 and V2, or between vertebrae V2 and V3).

As is particularly evident in the illustrations provided in FIGS. 11 and 12, the global joints between the attachment members 22 and the attachment extensions 20 of the spine attachment elements 12, 14, 16 permit the attachment members 22 ranges of motion relative to the respective attachment extensions 20, and relative to each other, sufficient to track even a complex helical bend, free from undue friction and/or binding. Further with reference to each of FIGS. 7-12, the relationship between the attachment members 22 and the elongated member 18 during the formation and/or relaxation of bends in the elongated member 18 is such as to permit and/or restrict relative axial/longitudinal relative movement between the attachment members 22 and the elongated member 18 along the axial direction of extension of the elongated member 18 (e.g., along the axis 24), as needed or as desired (e.g., depending on the desired function or functions of the spinal stabilization system 10, the needs of the particular patient, and/or the length of the elongated member 18, among other considerations).

It should be appreciated that numerous advantages are provided by the elongated member 18 and/or by devices such as the spinal stabilization device 10 that incorporate the elongated member 18 in accordance with the foregoing description to provide dynamic stabilization to the spine of a patient. Spine surgery patients whose conditions indicate that they would benefit from retaining at least some spinal motion in flexion, extension and/or axial rotation may benefit by being fitted with the dynamic spinal stabilization device 10 rather than undergoing procedures involving substantial immobilization as between adjacent vertebrae. The elongated member 18 (e.g., by virtue of its standard diameter sizing, substantial dimensional stability, and rigidity in tension and/or compression) is compatible with most rod attachment hardware presently being implanted in conjunction with lumbar fusion surgery and other spinal procedures, providing at least some basic similarity between the spinal stabilization device 10 and existing spinal stabilization devices, which similarity is advantageous insofar as it tends to simplify the process of seeking widespread industry acceptance and/or regulatory approval. The elongated member 18 is adaptable to pedicle screw attachment, allows for its use across two or more spinal levels, permits at least approximately seven degrees of lateral flexibility in spinal extension, spinal flexion, and/or spinal lateral bending as between adjacent spinal vertebrae, and allows for adjustable attachment points along the elongated member 46 to accommodate differing patient anatomies.

The helical shape of the elongated member 18 affords a substantially uniform, predictable level of bending flexibility (or, conversely, bending stiffness) in all lateral directions to facilitate smooth bending/flexure, and the diametrically opposed peripheral edge surfaces 30, 32 define a outer diameter compatible with the same conventional spine attachment hardware normally used in conjunction with solid, substantially laterally inflexible support rods. The elongated member 18 therefore includes far less mass and a much lighter weight than solid rods of similar diameters, thereby reducing the overall degree of modification of and/or impact on the spinal region affected by implantation. At the same time, sufficient material bulk is provided along the entire axis 24 of the elongated member 18 between the first and second inclined surfaces 34, 36 to maintain an adequate degree of rigidity against axial forces in compression and tension for purposes of spinal support/stabilization. The peripheral edge surfaces 30, 32 are generally contoured so as to define/adhere to a regular cylindrical shape, and can include multiple full radial turns across the axial extent of individual attachment members 22, facilitating secure coupling with hardware designed for coupling to cylindrically-shaped support rods. Further, the first and second inclined surfaces 34, 36 constitute flats across the diameter of the elongated member 18 which may be easily engaged and/or manipulated, whether for purposes of coupling to the attachment members 22 and/or pedicle screw heads, or by the practitioner during the process of implantation.

It should also be noted that the elongated member 18, and/or the dynamic spinal stabilization device 10 of which the elongated member 18 forms a part, are subject to numerous modifications and/or variations. For example, the elongated member 18 can be attached in many different ways to the attachment members 22 of the respective spine attachment elements 12, 14, 16, including embodiments wherein at least one of the attachment members 22 includes an axial hole through which the elongated member 18 either extends freely in the axial direction, or is clamped in place so as to prevent relative axial motion/translation, and embodiments wherein at least one of the attachment members 22 forms a hook (e.g., an incomplete hole) that includes no clamping means and therefore does not limit axial relative motion/translation of the elongated member 18. Many other variations in the spine attachment elements 12, 14, 16 are also possible, including the number of same provided in the context of the spinal stabilization device 10 (e.g., only two, four or more, etc.), as well as the method by which any or all are attached to their respective spinal vertebrae. The elongated member 18 can accordingly be shortened or lengthened, so as to be suitable for spanning a single pair of adjacent vertebrae, or more than three adjacent vertebrae. The first and/or second inclined surfaces 34, 36 need not necessarily be straight in transverse cross section, but can have different transverse geometries, such as curved, scalloped, tapered, convex, etc. Furthermore, the first and second inclined surfaces 34, 36 need not necessarily be parallel, or separated by a constant material thickness, but rather can diverge from a parallel orientation, and/or can be separated by differing material thicknesses, at different points along the axis 24 along which the elongated member 18 extends.

The elongated member 18 can be formed by a variety of different fabrication processes. For example, the elongated member 18 can be formed via a high-precision molding process. Excess material can be precisely removed from the radial periphery of the molded part by one or more suitable conventional processes, such as machining, grinding, polishing, etc., to arrive at the desired circular outer perimeter 26, the final diameter 28, and/or the first and second peripheral edge surfaces 30, 32. When formed by such a molding process, the structure of the elongated member 18 has minimal levels of internal stress (e.g., when in a straight configuration and prior to anterior, posterior, lateral, and/or helical bending). Accordingly, any stress that arises in the elongated member 18 as a result of anterior, posterior, and/or helical bending is generally not additive, and thus less likely to result in unusually high stress levels associated with fatigue and/or premature failure. Moreover, the helical shape is naturally associated with enhanced distribution of bending stress, further reducing the likelihood of damaging stress concentrations.

Rather than being molded as a fully-formed helical structure, the elongated member 18 can be formed from a flattened metal bar or strap that is subjected to axial twisting so as to produce a double helical structure. Such flattened metal bar or strap can include contoured lateral edge surfaces that are converted into a cylindrical shape when the flattened metal bar or strap is twisted. Alternatively, the lateral edge surfaces of such a flattened metal bar or strap can include substantially flat edge surfaces, which approximate a cylindrical shape, but which are more easily produced and/or dimensionally controlled. Alternatively, the elongated member 18 can be fabricating by machining an appropriately sized and shaped cylindrical blank as needed to produce the desired double helical geometry.

It will be understood that the embodiments of the present disclosure are merely exemplary and that a person skilled in the art may make many variations and modifications without departing from the spirit and scope of the invention. All such variations and modifications, including those discussed above, are therefore intended to be included within the scope of the present invention as described by the following claims appended hereto.

1. An elongated member configured and dimensioned for implantation adjacent the spine of a patient such that an axial span of said elongated member extends in an axial direction across at least one spinal level thereof and is adapted to promote efficacious spinal stabilization across said at least one spinal level, said axial span further manifesting a double helical geometry.

2. An elongated member according to claim 1, wherein said elongated member is configured and dimensioned for implantation adjacent the spine of the patient such that at least two axial spans of said elongated member extend in respective axial directions across respective spinal levels thereof and are respectively adapted to promote efficacious spinal stabilization across said respective spinal levels, each axial span of said at least two axial spans manifesting a double helical geometry.

3. An elongated member according to claim 1, wherein said axial span has a rod-like profile, and is adapted to be coupled to said spine of said patient via attachment to mounting members configured for coupling conventional support rods to said spine.

4. An elongated member according to claim 3, wherein said rod-like profile of said axial span includes a diameter in a range of from about 5.5 mm to about 6.35 mm.

5. An elongated member according to claim 1, wherein said axial span is adapted to permit pedicle screws to attach to said elongated member at multiple points along a length of said axial span so as to accommodate a range of different patient anatomies and spinal level heights.

6. An elongated member according to claim 1, wherein said axial span is substantially rigid as against axial forces arrayed in compression.

7. An elongated member according to claim 1, wherein said axial span is substantially rigid as against axial forces arrayed in tension.

8. An elongated member according to claim 1, wherein said double helical geometry of said axial span includes first and second peripheral surfaces disposed substantially diametrically opposite each other, and first and second helically inclined surfaces extending transversely between said first and second peripheral surfaces and oriented substantially parallel to each other.

9. An elongated member according to claim 1, wherein said double helical geometry of said axial span permits said axial span to bend along any and substantially all transverse directions while providing efficacious spinal stabilization across said spinal level during at least one of spinal flexion, spinal extension, spinal lateral bending, and spinal axial rotation.

10. An elongated member according to claim 1, wherein said axial span is adapted to provide efficacious spinal stabilization across said at least one spinal level during spinal flexion in which said at least one spinal level defines an anterior bend of at least approximately seven degrees.

11. An elongated member according to claim 1, wherein said axial span is adapted to provide efficacious spinal stabilization across said at least one spinal level during spinal extension in which said at least one spinal level defines a posterior bend of at least approximately seven degrees.

12. An elongated member according to claim 1, wherein said axial span is adapted to provide efficacious spinal stabilization across said at least one spinal level during spinal bending in which said at least one spinal level defines a lateral bend of at least approximately seven degrees.

13. A surgically implantable spinal support rod having an axial span extending in an axial direction so as to span at least one spinal level, said axial span manifesting a double helical geometry.

14. A spinal support rod according to claim 13, wherein said double helical geometry manifested by said axial span includes first and second peripheral surfaces disposed substantially diametrically opposite each other, and first and second helically inclined surfaces extending transversely between said first and second peripheral surfaces and oriented substantially parallel to each other.

15. A spinal support rod according to claim 13, wherein said axial span has a rod-like profile, and is adapted to be coupled to said spine of said patient via attachment to spine attachment devices configured for coupling conventional support rods to said spine.

16. An elongated member according to claim 15, wherein said rod-like profile of said axial span includes a diameter in a range of from about 5.5 mm to about 6.35 mm.

17. A kit for assembling a dynamic spinal support system, comprising:
a spinal support rod having an axial span extending in an axial direction so as to span at least one spinal level, said axial span manifesting a double helical geometry; and
a plurality of spine attachment devices attachable to said axial span so as to couple said spinal support rod to the spine of a patient across said at least one spinal level.

18. A kit for assembling a dynamic spinal support system according to claim 17, wherein at least one of said spine attachment devices is selected from the group consisting of a pedicle screw, a hook, a mounting plate and a cemented stem.
